SEQUENCE LISTING (6 sequences)

SEQ ID NO: 1 - DNA, 2325 bp, Artificial Sequence
  Description of Artificial Sequence Modified RdRp
atgcctcttctacacacactcaacacagcgctcgcagtgggactcctaggagccaggtactaccctgaggttcaaaccttcttggggctgcctgactacgtgggtcacatgaagaatgtagtacggtctgttttccagggatctgggctagtagtagtgtcctccgacacagtcggtgtcagggggacgtatagtaatagaggtcagataggtagtagtctcgggtgtatactagccgttccggatagcggggcggatatagaaatagacctagataggttggtaggaacggaagaggaagccacatcctgtttggtggaggcggtaggtagtaccgcagatgtccccaggaggagagttcgtcaaaaggggcggtttgctatgcatgccgtcaacgcagcaaagctgcacttttgtggcgtcccaaaacccactgaagcgaatcgactagcggtctcaaaatggcttgtccaatactgcaaagagagacatgtcgtagacagccacatcagaacgatagtcaatacggctcttcctagagtgttcacgcctgacgcggaagacattcaggtcgtgctggatttgcacagtgtaagagcacacgaccaccgcaacgccctagccgaagcaggcaaagtgcggaagtggtgggtcaatctcgcgatgcatcccatgactgggaggtcgtggtccagggcttggaggcgattatgccgactgcctgacgaccaggcgatctcttttgtccgcgggtgcttgcgggagctggtcgggagggagactcaaatctccaggggtgaaaacccggccatgcgcgtgttcccgttagcaaatcccccgaaggttcgacgcatcttccatatctgtggaatgggcaatggtttagactttggagtccacaacaactcactcaacaatttgagaagagggttgatggaaagagtcttttacgttgaagatgcgcagaagcaattgaaaccagccccccaaccgatcccagggattttcgggaagttgagtgggattcggagacgattggtcaggttggccggaaatcatacccctgtgcctcgggagaaatacccgtcgttctacaagggcaggagggccaccatataccaaaaggctttggattctctacatgacagaccggtatcccggaaggacgcagaactcaaaacattcgtgaaggcagaaaagatcaatttcacggctaagaaagacccggctccacgggtcatccagccgagggacccacgatataacattgaggttgggaaatacttgaaaccgtacgagcaccatttatatcgggcaattgacgctatgtggggtgggcccactgtgctgaaaggatacgatgtgggggagcttggaaacattatgagtaacacctgggataaattccggaaaacgtgtgcgataggatttgacatgaagagattcgaccagcacgtatccgtggacgccctacgatgggaacacagtgtatacaacgcgggctttaactgtcccgagttggcacagctgctaacttggcagttgaccaacaagggagttgggagagcctccgatggctttatcaaataccaagttgatggttgtcgcatgtccggagatgttaacacagccttgggcaactgcctactggcttgctctatcaccaagtacttaatgaagggaatcaaatgcaaattaatcaacaatggagacgattgtgtgctgttcttcgaagctgatgaagtcgacagggtgcgcgaaaggctgcatcattggatcgactttgggtttcaatgcatagcggaagaaccacaatacgaattggagaaagttgaattttgccagatgtcccctattttcgatggtgaagggtgggtcatggtcagaaacccccgtgtgagcctctccaaggacagctacagcaccacacaatgggcgaatgagaaagatgcagccagatggttggctgccatcggagagtgtggcttggctattgcaggtggcgtaccagtgttacaatcatattattcttgcctgaagaggaattttggacccctggccggggactacaagaagaagatgcaagatgtttcctttgatagtggattctacaggttatccaagaacgggatgaggggcagcaaagacgtgtcccaagatgctaggttcagcttttaccgggggttcggctacactccagacgagcaggaagcgcttgaggagtactacgacaacctcgaactgctctgtgagtgggaccccacgggatataaagaagaacttagtgatagatggatcctgaacgaattccctacaactctctaa

SEQ ID NO: 2 - protein, 504 aa, Artificial Sequence
  Description of Artificial Sequence Modified RdRp
MRVFPLANPPKVRRIFHICGMGNGLDFGVHNNSLNNLRRGLMERVFYVEDAQKQLKPAPQPIPGIFGKLSGIRRRLVRLAGNHTPVPREKYPSFYKGRRATIYQKALDSLHDRPVSRKDAELKTFVKAEKINFTAKKDPAPRVIQPRDPRYNIEVGKYLKPYEHHLYRAIDAMWGGPTVLKGYDVGELGNIMSNTWDKFRKTCAIGFDMKRFDQHVSVDALRWEHSVYNAGFNCPELAQLLTWQLTNKGVGRASDGFIKYQVDGCRMSGDVNTALGNCLLACSITKYLMKGIKCKLINNGDDCVLFFEADEVDRVRERLHHWIDFGFQCIAEEPQYELEKVEFCQMSPIFDGEGWVMVRNPRVSLSKDSYSTTQWANEKDAARWLAAIGECGLAIAGGVPVLQSYYSCLKRNFGPLAGDYKKKMQDVSFDSGFYRLSKNGMRGSKDVSQDARFSFYRGFGYTPDEQEALEEYYDNLELLCEWDPTGYKEELSDRWILNEFPTTL

SEQ ID NO: 3 - DNA, 402 bp, Artificial Sequence
  Description of Artificial Sequence SatC sequence, sense orientation
cccgggataactaagggtttcatacgataccacgcaactaatgcagaacacccattgccctaaacttgactgatgacccttacgtaggccgggggggtttaaccatggtgggttgtgaaggcgggagttcccatcaagtacgggggcgtgaaacctgacagtatcccactcgaaagagtcttctcctctctcttatattaagaaaaggaaacaaaacccccaggtcgctttattttgacctgtgttagggaccaaaaacggtggcagcactgtctagctgcgggcattagactggaaaactagtgctctttgggtaaccactaaaatcccgaaagggtgggctgtggggaccttccgaactaaaagatagcctccctcctcgcgggggggggcctgcccggg

SEQ ID NO: 4 - DNA, 402 bp, Artificial Sequence
  Description of Artificial Sequence SatC sequence, antisense orientation
cccgggcaggcccccccccgcgaggagggaggctatcttttagttcggaaggtccccacagcccaccctttcgggattttagtggttacccaaagagcactagttttccagtctaatgcccgcagctagacagtgctgccaccgtttttggtccctaacacaggtcaaaataaagcgacctgggggttttgtttccttttcttaatataagagagaggagaagactctttcgagtgggatactgtcaggtttcacgcccccgtacttgatgggaactcccgccttcacaacccaccatggttaaacccccccggcctacgtaagggtcatcagtcaagtttagggcaatgggtgttctgcattagttgcgtggtatcgtatgaaacccttagttatcccggg

SEQ ID NO: 5 - DNA, 3404 bp, Artificial Sequence
  Description of Artificial Sequence Vector pIJO-20
gggcgaattgggcccgacgtcgcatgctcccggccgccatggccgcgggattcccgggcaggcccccccccgcgaggagggaggctatcttttagttcggaaggtccccacagcccaccctttcgggattttagtggttacccaaagagcactagttttccagtctaatgcccgcagctagacagtgctgccaccgtttttggtccctaacacaggtcaaaataaagcgacctgggggttttgtttccttttcttaatataagagagaggagaagactctttcgagtgggatactgtcaggtttcacgcccccgtacttgatgggaactcccgccttcacaacccaccatggttaaacccccccggcctacgtaagggtcatcagtcaagtttagggcaatgggtgttctgcattagttgcgtggtatcgtatgaaacccttagttatcccgggaatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 6 - DNA, 5451 bp, Artificial Sequence
  Description of Artificial Sequence Vector pIJO-79
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgcgaaggcgaagcggcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgggtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggaaagctggtacgcctgcaggtaccggtccggaattgggataactaagggtttcatacgataccacgcaactaatgcagaacacccattgccctaaacttgactgatgacccttacgtaggccgggggggtttaaccatggtgggttgtgaaggcgggagttcccatcaagtacgggggcgtgaaacctgacagtatcccactcgaaagagtcttctcctctctcttatattaagaaaaggaaacaaaacccccaggtcgctttattttgacctgtgttagggaccaaaaacggtggcagcactgtctagctgcgggcattagactggaaaactagtgctctttgggtaacctagagtcgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtaaccactaaaatcccgaaagggtgggctgtggggaccttccgaactaaaagatagcctccctcctcgcgggggggggcctgcccgggtcggcatggcatctccacctcctcgcggtccgacctgggcatccgaaggaggacgcacgtccactcggatggctaagggaggggcggggatccggctgctaacaaagcccgaaaggaagctgagttggctgctgccaccgctgagcaataactagcataaccccttggggcctctaaacgggtcttgaggggttttttgctgaaaggaggaactatatccggatccactagttctagaggatccaagcttacgtacgcgtgcatgcgacgtcatagctcttctatagtgtcacctaaattcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

(19) United States
(12) Patent Application Publication
(10) Pub. No.: US 2004/0265821 A1
(43) Pub. Date: Dec. 30, 2004

(54) Rna amplication system using plant components in animal cells

(76) Inventors: Volker Sandig, Berlin (DE); Ingo Jordan, Berlin (DE)

Correspondence Address: NEEDLE & ROSENBERG, P.C., SUITE 1000, 999 PEACHTREE STREET, ATLANTA, GA, 30309-3915, US

(21) Appl. No.: 10/485,281

(22) PCT Filed: Jul. 29, 2002
(86) PCT No.: PCT/DE02/02863

(30) Foreign Application Priority Data
Jul. 30, 2001 (DE) 101 37 444.5

(51) Int. Cl. C12Q 1/68; C12P 19/34; C12N 15/86

(52) U.S. Cl. 435/6; 435/91.2; 435/456

(57) ABSTRACT

The invention relates to a method and a novel system for the constitutive or inducible, stable or transient intracellular amplification of foreign RNA in an animal cell or organism. The system is based on an autonomous, RNA-dependent RNA amplification by the expression of the RNA-dependent RNA polymerase (RdRP) of a plant virus in animal cells. The amplification is initiated by an RNA transcript, (primary transcript), comprising the cis-active sequences for said RdRP. The amplified RNA can act as mRNA for protein synthesis, as effector RNA, (for example as anti-sense RNA against specific mRNA or viral RNA molecules, as a ribozyme against cellular RNA molecules or recombinant structure RNA in ribosomes or spliceosomes), or as genomic RNA for the production of recombinant viruses. In vivo applications include gene therapy, vaccination and therapeutic vaccination.

The invention relates to a novel system for the constitutive or inducible, stable or transient intracellular amplification of foreign RNA in an animal cell or organism. The system is based on an autonomous, RNA-dependent RNA amplification by the expression of the RNA-dependent RNA polymerase (RdRP) of a plant virus in animal cells.

The amplification is initiated by an RNA transcript (primary transcript) comprising the cis-active sequences for said RdRP (for a list of abbreviations, see after the Examples). The amplified RNA can act as mRNA for protein synthesis, as effector RNA (for example as antisense RNA against specific mRNA or viral RNA molecules, as a ribozyme against cellular RNA molecules or recombinant structural. RNA in ribosomes or spliceosomes), or as genomic RNA for the production of recombinant viruses. In vivo applications include gene therapy, vaccination and therapeutic vaccination.

### BACKGROUND OF THE INVENTION

Expression of foreign genes in animal cells usually is accomplished by transfection of the open reading frames as cDNA (complementary DNA, copy DNA) into the cell. This cDNA can be a component of plasmid DNA flanked by cis-active sequences such as promoters and polyadenylation sites which allow synthesis of mRNA (messenger RNA) in the target cell.

Transfection of plasmid DNA into a host cell usually causes only transient expression of the foreign genes: due to cell division and degradation of the introduced DNA, the daughter cells have less and less plasmids, which are finally lost within weeks [Ausubel et al. (ed) 2000, Current Protocols in Molecular Biology, John Wiley & Sons].

For this reason, to accomplish stable gene expression, foreign genes either need to be integrated into the chromosome of the host cell, or their replication must be coupled to cell division by episomal functions in the plasmid backbone. If the foreign gene is toxic towards the cell, the promoter must be blocked during the establishing of the cell line. Several systems are available for such an inducible inactivation of promoters. However, all these systems share some basal activity of the controlled promoter so that a measurable background expression of mRNA may occur [Ausubel et al. (ed) 2000, Current Protocols in Molecular Biology, John Wiley & Sons].

Currently, three main parameters are adjusted to achieve high level expression of recombinant genes: (1) strength of the promoter which causes the synthesis of the mRNA transcripts; (2) translation capability of the resulting mRNA, mainly through optimization of RNA export into the cytoplasm, of the initiator tRNA environment (tRNA=transfer RNA) and of the codon frequency in the open reading frame; and (3) the number of. DNA copies either by one chemically induced gene amplification of integrated copies, or the number of plasmid molecules through SV40-mediated or episomal accumulation. Current state of the art has the first two parameter optimized to a very high degree and appears to substantially exhaust the efficiency of conventional host cells. Increasing the number of DNA copies beyond a certain threshold value results only in minor increases in expression yield. Furthermore, amplification of integrated DNA is accompanied by random mutagenesis of the genotype and thus unpredictable in its effect on stability of the host cells [Snapka et al. 1997, Cell. Prolif. 30, 385-99].

Other approaches make use of recombinant viruses allowing expression of cDNA in high amounts and often optimum transduction efficiencies. Well-known systems mainly rely on vaccinia, herpes, adeno-, alpha-, retro- and parvo-viruses (adeno-associated viruses); more recent developments try to use polio-, flavi- and rhabdo-viruses (rabies viruses). In some approaches the foreign gene interrupts a region which is irrelevant to the viral replication in cell culture, resulting in replication competent recombinant viruses. However, the foreign gene may also disrupt essential regions for viral replication or morphogenesis. Such viruses express the foreign gene, but cannot form progeny viruses without trans-complementing the lacking factors.

Compared to transfection of expression plasmids, the advantage of viral systems resides mainly in their higher transduction efficiency and, for replication-competent systems, their higher expression rate due to virus multiplication and inhibition of the host cell's biosynthesis, so that more resources are available for viral expression. However, a stable transduction is not possible in this case since the high level expression of the foreign gene is coupled to productive infection. Although for some systems, for example, vaccinia viruses, herpes viruses, adenoviruses and alpha viruses, there are virus mutants with attenuated cytopathogenicity, activation of the virus usually results in death of the host cell. Furthermore, such preparations bear the inherent danger of contamination with helper virus or emergence of recombinant viruses with unpredictable properties. This aspect is important as the above mentioned systems are usually derived from human pathogens. Accidental infections in laboratories from working with various systems and the death of one laboratory worker from Semliki forest virus (an alphavirus) have been described [Willems et al. 1979, Science 203, 1127-9]. Attenuated viruses may also develop a higher pathogenic potential in immunosuppressed patients [Lustig et al., 1999, Arch Virol 144, 1159-71; Centers for Disease Control and Prevention 2001. MMWR 50 NO. RR-10].

With viruses with a DNA replication phase, there is also the risk of integration of viruses into the genome of the cell; especially for retroviruses, integration is even part of the viral replication. Since integration may change the phenotype of the host cell and may cause transformation and thus cancer in gene therapy, it is an undesired attendant phenomenon of some approaches.

Compared to transfection of expression plasmids, many viral systems are clearly more demanding in methodology in terms of the preparation of the vectors. Vaccinia, adeno- and herpes viruses are not amenable to direct manipulation due to their large genomes and require recombination events between recipient and transfer vectors. Furthermore, even in less complex systems such as flavi- and alphavirus vectors, the expression of the foreign gene requires the formation of heteromeric viral protein complexes and replication events that are not yet fully understood [Khromykh et al., 1999, J Virol 73, 10272-80; Perri et al. 2000, J Virol 74, 9802-7].

In another approach, unmodified RNA is synthesized in the cytoplasm through recombinant T7 RNA polymerase (free of cap structure and without polyadenylation). For an efficient translation, these transcripts must be provided with IRES elements (IRES=internal ribosomal entry site) [Elroy-Stein, 1989, PNAS 86, 6126-30]. The template usually is a plasmid DNA which is only transiently available in the cytoplasm after transfection as it migrates to the nucleus. Through artificial nuclear localisation sequences, the T7 RNA polymerase could be directed into the same cellular compartment in which these templates accumulate. However, these approaches result in inefficient protein expression since the synthesized RNA probably is not directed onto the export pathway for mRNA molecules and thus reaches the cytoplasm, the site of protein biosynthesis, only in a low number [Lieber et al.; 1993, Meth. Enzym. 217, 47-66].

### DESCRIPTION OF THE INVENTION

The object of the present invention is to provide a system and method for the amplification of nucleic acids in animal cells.

This object was achieved by a system in the form of a combination consisting of an RNA-dependent RNA polymerase (RdRp) of a plant virus and an RNA promoters or cis-active signals. This system for the amplification of RNA in animal cells, except for human parent cells, but including mammal, including human, insect and amphibian cells, is based on that the RdRp of a plant virus is brought in animal cells together with a substrate RNA which contains one or more cis-active signals which are recognized by the RdRp. The gene for the RdRp may be derived from plant cells or a plant virus, preferably from the Tombusviridae family. The substrate RNA can be synthesized in the host cell as a primary transcript or introduced into the animal cell from outside.

The system according to the invention and the method for the amplification of nucleic acids in animal cells comprises introducing an RNA-dependent RNA polymerase (RdRp) of a plant virus into the animal cells.

Significant innovative steps of the system described include (1) expression of a functional plant replicon in an animal cell; (2) a two-fold block for the silencing of genes because the primary transcript may bear genes in a non-coding orientation so that conversion into a coding mRNA is effected only in the presence of the RdRp, which can also be regulated; (3) reduction of RNA-dependent RNA amplification to a polymerase and the related primary transcript. Viral structural genes or helper viruses are not required. (4) The polymerase and primary transcript can be expressed independently of each other.

With the solution according to the invention, it is surprisingly possible to successfully achieve amplification of nucleic acids in animal cells with as small as possible a number of components, preferably directly in the cytoplasm, and free from components which could be potentially infectious for animals.

The present invention is different from patent applications such as WO 99/02718 A1 in that a plant virus system is transferred onto animal models in a surprisingly reduced way. The invention is also different from patent applications describing the use of isolated polymerases or RdRps for amplification of nucleic acids (such as WO 99/49085 A1 and U.S. Pat. No. 5,556,769) because here the use of such an amplification system is realized in living cells (rather than cell extracts), so that the present invention may also be employed in bioreactors and, for example, the amplified RNA can not only be translated into a protein or proteins, but post-translational modifications, such as phosphorylation, glycosylation or proteolytic maturation are also possible.

The system according to the invention is based on the adaptation of the replication of the plant virus TCV (turnip crinkle virus; Tombusviridae) in animal cells. The system expands the potential of conventional approaches by the unusual use of plant virus enzymes in mammal cells. It may also be implemented with other viruses of the Tombusviridae such-as carnation mottle virus, or with other virus families with similar genomic organization, such as Bromoviridae, or even segmented viruses such as Potyviridae [Mayo & Pringle, 1998, in J. Gen. Virol. 79, 649-57]. Another desirable an extremely important advantage over all conventional systems is the fact that no pathogens infecting humans and other vertebrate animals are known to exist among the Tombusviridae and some other plant virus families.

As this manuscript was submitted biosynthesis of a plant RdRp has been proposed for purification also in mammalian cells (U.S. Pat. No. 6,218,142 B1 2001). However, it does not intend RNA amplification and foreign gene expression by a. recombinant RdRp, but inactivation or over-expression of an endogenous RdRp, preferably from uninfected tomatoes, which can inhibit foreign gene expression via a gene silencing mechanism in plant cells and possibly also in mammal cells. Further, the expression of a recombinant RdRp in foreign cells for mediating gene silencing is intended. However, in the present application, a plant virus RdRp which is not related to the RdRp used in the specification U.S. Pat. No. 6,218,142 B1 2001 is to be employed, preferably for the high level expression of coding RNA.

The genome of the viruses from the Tombusviridae family consists of a single RNA strand of about 4500 nucleotides in a coding orientation. The carnation mottle virus of this family has been shown to contain a cap structure at the 5′ terminus of the genomic RNA. Replication of Tombusviridae does not require any host cell factors or post-translational modifications of viral proteins. As clearly opposed to related alphaviral systems, the promoter elements are located at the termini of the genomic RNA; internal initiation of transcription for the synthesis of subgenomic RNA molecules is not required.

For the present application, the viral protein recombinantly expressed is RNA-dependent RNA polymerase (RdRp), i.e., in contrast to some other systems, without the necessity of complementation by a helper virus. As an additional advantage compared to adenoviral or polioviral systems, covalent modification of the genomic nucleic acid by terminal proteins is not necessary. As an educt for the intracellular amplification of RNA, a primary transcript is employed which bears the corresponding recognizable cis-active signals, for example, viral promoters (VP) or artificial promoters or terminators, of some hundred nucleotides. Further, it is a novel feature that no viral genes are required on the primary transcript. In addition, packaging into viral capsids also is not required for amplification so that the primary transcript can accept very large foreign genes or inserts.

Tombusviridae dispose of different promoters with varying expression strengths. Because the primary transcript and RdRp can be expressed independently of each other, it is additionally possible, in contrast to as yet known systems, to effect co-expression of groups of genes through several different primary transcripts. This modular setup allows a novel and surprising flexibility in the performance of the expression, co-expression or high level expression of proteins, as well as one-sided amplification or replication of RNA (also mRNA provided with a cap structure). In spite of this high flexibility, the preparation of the expression cassettes can be achieved by direct, simple procedures familiar to the skilled person without the requirement for recombination or complementation by helper viruses.

In a preferred embodiment, the primary transcript is synthesized directly in the cell, either in the cytoplasm or in the nucleus, by cellular polymerases, such as RNA polymerase II, or by recombinant polymerases, such as RNA polymerase T7. Alternatively, the primary transcript may also be synthesized in vitro, for example, with bacteriophage T7, Sp6 or T3 RNA polymerase, and then transfected into the cell.

In a preferred embodiment of the system, the RdRp is provided in trans either by induction or by constitutive expression from its own expression cassette. The primary transcript is expressed independently of the RdRp. On the primary transcript a VP follows a foreign gene. Both the foreign gene and the VP are in non-coding orientation (see FIG. 1). In this way, it is ensured that the foreign gene cannot be translated even if the silencing of the expression cassette for the primary transcript should be incomplete, thus providing an additional barrier in the expression of toxic gene products. To our knowledge, such an approach for control of gene expression has not been described previously. The primary transcript is the substrate for the RdRp for synthesis of an antisense copy. However, this copy carries the gene in coding orientation, so that translation may occur now. Since the RdRp of the Tombusviridae probably provides its transcripts with a cap structure, no IRES element is required for translation. However, in other applications, the translation efficiency may be changed or made controllable by inserting an IRES upstream. The step of substrate recognition of the primary transcript by the viral RdRp results in an RNA-dependent amplification of an mRNA and thus in a high level expression of a foreign gene. A great novel advantage of this system over conventional viral expression methods is controllability: If the expression of the RdRp or of the primary transcript is blocked, the system is shut down.

In another embodiment, the VPs flank the foreign gene (FIG. 1). Such a primary transcript is replicated and amplified. The coding transcripts can now serve as a template for the synthesis of further copies of the primary transcript. These copies in turn bring about further coding transcripts, whereby an exponential amplification of specific transcripts is achieved as a precondition for a clearly enhanced foreign gene expression. Contrary to present persisting viral systems, this system still can be switched off even at maximum expression levels for the foreign gene by blocking expression of the RdRp. In further embodiments of this system, natural or modified satellite RNA can be used as a primary transcript. In yet further embodiments, the VP for the non-coding transcripts can be adjusted clearly weaker or stronger than the VP which generates the coding transcripts by appropriately selecting wild-type promoters (for example, promoters in satellite RNA [Carpenter & Simon, 1998, Nucl. Acids Res. 26, 2426-32; and Simon et al. 1988, EMBO J 7,-2645-51] or on the genomic RNA [Carrington et al. 1989, Virology 170, 219-26]) or by mutagenesis in cis-active sequences. Thus, another modulation of expression efficiency is possible.

In another embodiment, the VPs flank both the RdRp and the foreign gene in independent transcripts. This system amplifies and replicates the expression of both the RdRp and the foreign gene. Depending on the choice and strength of the VPs by the operator, the system may take any of three pathways: (1) The host cell is overwhelmed by the high level expression and dies. (2) An artificial replicon which is only RNA-based is formed and maintained in the cell. (3) Replication gradually declines if the turnover of the RNA and RdRp cannot be compensated by the replication strength of the VPs.

In aspects of the above described embodiment, the primary transcript encodes the RdRp and a foreign gene and additionally contains functional viral promoters at both termini. Such a primary transcript is brought into the cell or into an organism as an RNA in coding orientation with respect to the RdRp to initiate replication. In this way, the transduction of cells is achieved without employing a DNA phase, whereby the risk of unintended modifications in the genome of the host cell is reduced. Since the protein expression and replication are effected in the cytoplasm and thus a nuclear localization is not required, resting cells can also be transduced by this system.

In another embodiment, an RNA segment which displays an action in the cell without translation, such as ribozymes, ribosomal RNA or antisense constructs or a genomic or subgenomic RNA of another virus, is used in the system in place of the foreign gene. Thus, such functional RNA can be amplified by the system and its effect in the cell enhanced. In the case where a genomic or subgenomic RNA of another virus is used, effects of viral infection are simulated without using the replication mechanism of the respective virus. This yields a particularly safe and highly attenuated system. Alternatively, the system is used for the preparation of recombinant viruses.

In aspects of the above embodiments, the activity of the RdRp is modulated by utilizing the temperature optimum by appropriately changing the culture temperature of the mammal cells.

In further aspects of this system, primary transcripts are modified such that substrate recognition by the RdRp is either improved or impaired. Methods for achieving this may include mutagenesis in cis-active sequences, insertion and deletion. Further, the size of the primary transcript may be varied. By selecting different or identical promoters or promoters and satellite RNAs of related viruses, transcripts having different replication properties can be generated. Internal promoters on the primary transcripts can be employed for the synthesis of smaller derivative transcripts. The position and number of the foreign genes can be varied. The number of different primary transcripts can be varied.

In further aspects, the translation capability and half-life of the transcripts formed are improved by artificial polyadenylation stretches or by insertion of elements that modify the interaction with ribosomes, such as stem loops, IRES elements, such as those from encephalomyocarditis virus or polio virus [Pestova et al. 2001 in Proc. Natl. Acad. Sci. USA 98, 7029-36], shunt donor and acceptor, such as those from cauliflower mosaic virus [Fütterer et al. 1993 in Cell 73, 789-802] or translational enhancers, such as the leader from the cellular protein p27 [Miskimins et al. 2001 in Mol. Cell. Biol. 21, 4960-7].

In other aspects of the system, the primary transcript is equipped with recognition signals which may cause packaging by viral capsid proteins or ssRNA binding proteins, or which enable processing of the transcripts by endonucleases, RNA-editing enzyme or by the splice machinery. In related aspects, the recognition signals can be designed in such a way that only the amplified transcripts having a defined orientation are recognized.

In a preferred application of the system, the greater inherent error rate of RNA-dependent RNA amplification is used for broadly scattered mutagenesis and for finding particularly suitable mutations in RNA segments which may code for a gene or else represent regions which display their actions as a non-translated RNA, such as the viral promoters of the system themselves, ribozymes, antisense RNA or structural RNA in ribosomes or spliceosomes. In an especially interesting application of the system, the amplified transcripts may encode a foreign gene whose activity is monitored as a function of time until particularly suitable mutations in the foreign gene or in the viral promoters have been found. Since the flanking sequences are known, the corresponding sequence can be obtained very simply and selectively via PCR. In another application of the system, the foreign gene may be replaced by a protein having an undesirable cytotoxicity; in a time study, replicons whose host cells grow faster than neighboring cells can be rescued for finding mutants having a particularly low cytotoxicity.

In another preferred application of the system, the RdRp may be coupled to a recognition sequence for a controllable protein, such as inhibitor kappa B (IkB) or a modified estrogen receptor. Binding by the cellular protein results in a reversible blocking of the RdRp activity which is released upon dissociation of the factor. For example, the active RdRp could cause transcription of the antisense RNA for a foreign gene, which can now be translated. In this way, an intracellular measuring system for particular cellular interactions can be established.

The features of the invention can be seen from the elements of the claims and from the description, protection being requested by this document for both individual features and several advantageous embodiments in the form of combinations. The features are composed of novel elements, such as the use of plant virus enzymes (RNA-dependent RNA polymerase (RdRp)) in mammal cells and the separation of transcripts for RdRp (having the activity of a replicase) and substrate RNA, and known elements, such as RNA amplification and protein synthesis, whose combination leads to the novel advantageous system according to the invention.

The spirit of the invention resides in a system for the amplification of RNA in animal cells using an RNA-dependent RNA polymerase (RdRp) of a plant virus and an RNA which contains promoters or cis-active signals. The system according to the invention relates to animal cells including mammal, including human (with the exception of embryonal stem cells), insect, worm and amphibian cells. It is characterized by containing the RdRp of a plant virus whose gene is obtained from plant cells or a plant virus and introduced into animal cells, and RNA which contains a promoter or promoters or cis-active signals recognized by the RdRp and which is synthesized as a primary transcript or introduced into the animal cell from outside.

Further, the method according to the invention is based on the fact that the RdRp as a transcript contains an RdRp gene which is introduced into the animal cells.

For the intracellular amplification of RNA, the method/system according to the invention uses an RdRp:

which is encoded by a separate transcript separately from its promoter; or

whose gene is part of the primary transcript; or

whose gene is part of its own transcript;

and a primary transcript

whose properties as a substrate are modified by mutagenesis; and/or

whose foreign gene and at least one promoter are in antisense orientation so that the foreign gene cannot be expressed without an RdRp; and/or

which contains IRES elements, shunt donor and acceptor, or translation enhancers for improving the gene expression;

wherein the amplified RNA

contains at least one polyadenylation tract; and/or

is processed by a ribozyme; and/or

contains signals for packaging into a viral envelope and is packaged into viral envelopes; and/or

codes for a gene or several genes; and/or

codes for genes in different orientations; and/or

represents an RNA which displays its action in cells without translation, such as ribozymes, ribosomal RNA or antisense constructs; and/or

codes for a genomic or subgenomic RNA of another virus and thus contains an RdRp;

whose activity is modulated by changing the culturing temperature;

whose toxicity is reduced by mutagenesis in combination with selection.

The system according to the invention contains a primary transcript which is synthesized in vitro by a cellular polymerase or by a bacterial RNA polymerase, such as T7, SP6 or T3, or within the cell. The promoters on the primary transcript are derived from a plant virus, i.e., a member of the Tombusviridae family, especially turnip crinkle virus.

The method according to the invention is further based on the occurrence of the amplification of an RNA which codes for a foreign gene, acts as an antisense transcript of a cellular transcript, serves as a genomic RNA for the preparation of recombinant viruses, or has itself enzymatic activity.

The strength of naturally occurring promoters for the RdRp is changed by mutagenesis. The foreign gene and at least one promoter of the RdRp in the primary transcript are in antisense orientation, wherein the foreign gene cannot be expressed without the RdRp, but is activated by expression of the RdRp.

The RNA amplified by the RdRp bears IRES elements, shunt donor and acceptor and/or translation enhancers for improving the expression of the foreign gene. It contains at least one polyadenylation tract and is further characterized by:

being processed by a ribozyme; and/or

containing signals for packaging into a viral envelope and being packaged into viral envelopes.

The RNA contains two equal or different promoters in opposite orientations which initiate the synthesis of the two RNA strands to thereby induce an enhanced amplification.

The expression of the primary transcript, the RdRp or both is regulated by a system based on cellular RNA polymerase II.

The enzymatic activity of the RdRp is promoted, reduced or switched off by changing the culturing temperature.

The application according to the invention serves for finding favorable mutations in the RdRp or in the foreign gene. The favorable mutations in turn cause an improved replication of the system, an improved performance of the foreign gene, and/or a lower toxicity of the foreign gene or the RdRp.

The system according to the invention is designed to be switched on or activated by cellular events. The activation in turn:

causes expression of a foreign gene, including a reporter gene; and/or

is effected through a fusion protein at the RdRp; and/or

serves for the detection of signal transduction pathways and the translocation of intracellular factors; and/or

is effected by the entering of a diffusible substance or a toxin or through infection of the host cell.

It is essential to the invention that:

the polymerase and the promoters are derived from a plant virus;

the polymerase from a member of the Tombusviridae family is used, especially the polymerase of turnip crinkle virus or carnation mottle virus;

a modified satellite RNA of turnip crinkle virus or a modified genomic RNA of turnip crinkle virus is used as the amplified RNA;

the system is applied in vivo, i.e., in humans, mammals or insects.

The invention relates to a novel system and a novel application for the constitutive or inducible, stable or transient intracellular amplification of foreign RNA in an animal cell or organism. The system is based on autonomous RNA-dependent RNA amplification by the expression of the RNA-dependent RNA polymerase (RdRp) of a plant virus in animal cells. The initiation of the amplification is effected by an RNA transcript (primary transcript) with the cis-active sequences for this RdRp. The amplified RNA may serve as an mRNA for protein synthesis, as an effector RNA (for example, as antisense RNA against certain mRNA or viral RNA molecules, as a ribozyme against cellular RNA molecules, or as recombinant structural RNA in ribosomes or spliceosomes), or as a genomic RNA for the preparation of recombinant viruses. The in vivo applications include gene therapy, vaccination and therapeutic vaccination.

The test system (test kit) according to the invention includes two components: component (1) is a cell line which bears the gene for the RdRp stably integrated in its genome and expresses this gene constitutively or, under the control of a controllable promoter, such as the tetracyclin system, only upon induction. The preferred cell line is easily transfectable, so that the foreign gene with the cis-active signals for the RdRp can be simply introduced into these cells. Component (2) is a collection of expression plasmids for the primary transcript, where in the preferred plasmids the cis-active signal sequences for the RdRp are combined with a region having several restriction sites for various restriction enzymes, so that the insertion of foreign genes is as simple as possible. The expression plasmids are equipped with different arrangements of the cis-active sequences, so that amplification with different strength or amplification coupled with replication are possible depending on the selection of the expression plasmid.

In addition, the expression plasmids are equipped with promoters of different strengths and polyadenylation signals for the expression of the primary transcript in the target cell. In addition, the expression plasmids have a bacterial promoter at the beginning and a suitable restriction site for a restriction enzyme at the end of the cassette for the primary transcript, so that the primary transcript can be synthesized in vitro and can be transfected into the cell as an RNA rather than plasmid DNA. In addition to the terminal restriction site for a restriction enzyme, the expression plasmid bears a transcription terminator for the bacterial polymerase. Thus, the expression plasmid can express primary transcripts also in the eukaryotic cell through co-transfected bacterial polymerases. For this purpose, the gene for the bacterial polymerase may also be expressed by its own cassette on the expression plasmid, so that co-transfection is not necessary.

Another test kit according to the invention consists of a cell line that carries both the RdRp and the expression cassette for the primary transcript stably integrated in its genome. However, the RdRp is under the control of an inducible promoter which is to respond to a test substance of the user. The primary transcript bears both a gene for the RdRp and for a reporter gene in a replicable and amplifiable combination, i.e., cis-active signals for the RdRp on both sides of the reporter gene and the gene for the RdRp on the primary transcript. This extremely sensitive system results in a very quick and strong expression of the reporter protein when the cell is exposed to the test substance. In a preferred application, the inducible promoter responds to infection by another virus, for example, human immunodeficiency virus, and thus allows for quick clinical diagnostics. In another preferred application, the inducible promoter for the RdRp responds to the presence of heavy metal ions and thus enables a fast and quantifiable detection of environmental loads (“biosensor”).

The invention shall be further described by Figures and Examples without being limited thereby.

### EXAMPLES

Total RNA was extracted from a TCV virus inoculate in the form of dried infected leaves (DSMZ PV-0293; Deutsche Sammlung von Mikroorganismen und Zellkulturen of Braunschweig, Germany) using acidic phenol. (Trizol, Gibco BRL), denatured at 80° C. for 10 min in the presence of random hexamer or SatC-specific primers, and converted into cDNA by incubation with Superscript II reverse transcriptase (Gibco BRL) for 60 min at 42° C. Design of primers for the PCR amplifications and subsequent cloning was based on GenBank sequences #M22445 for TCV and #X12750 for the satellite RNA C (SatC).

1. Cloning of the RdRp

The gene for the RdRp (TCV 88 kD protein) was amplified out of the above cDNA mixture with primers i113 (ataccggtatgcctcttctacacac) and i114 (tagcggccgcttagagagttg) using Taq (Qiagen GmbH, Max-Volmer-Straβe 4, D-40724 Hilden) in a PCR with 10 cycles of

94° C. for 10 sec (denaturing step),

56° C. for 30 sec (annealing) and

68° C. for 2 min (polymerization), followed by additional 20 cycles of

92° C. for 10 sec,

56° C. for 30 sec and

68° C. for 2 min, where this polymerization step was extended with each cycle by 20 sec.

The primers contain the target sequences for restriction enzymes Age I and Not I, so that specific insertion into the vector pEGFP-N1 (Clontech Laboratories, Inc., 1020 East Meadow Circle, Palo Alto, Calif. 94303-4230, USA) should be possible, thus replacing the gene for GFP (green fluorescent protein). The expression of the 88 kD protein is then placed under the control of the hCMV IE promoter.

In the gene for the 88 kD protein, the most significant deviations were found in three independent clones from sequence #M22445: (a) Of two published internals stop codons, the first stop codon at position 209 does not exist. Instead of aga/gtc/aga/*TA/Ggt, the sequence is aga/ggt/cag/aTA/Ggt in these experiments. (b) In the gene were found an insertion at position 1137 and a deletion at position 1156. This results in a transient shift of the reading frame with respect to the published sequence, so that the amino acid sequence reads LDSLHDRPVSR instead of LDLYMTCRLSR. (c) It was particularly surprising that deletion at position 1156 from accggCt to accggt created an Age I restriction site. This effect was observed in all PCR fragments and complicated cloning considerably because the strategy was based on the published sequence. Cloning succeeded by digestion with Not I and a partial digestion which left the Age I restriction site in the gene intact; the thus treated amplification product was cloned into the Age I and Not I sites of the vector pEGFP-N1, whereby the reading frame of EGFP is replaced by the RdRp to form plasmids pIJO-03, pIJO-18 and pIJO-19. These three clones served for the determination of the gene sequence for the 88 kD protein and form the basis for the further work (summarized in FIG. 2).

According to the literature [White et al., 1995 in Virology 211, 525-34], Tombusviridae express the 88 kD protein by suppression of an internal amber stop codon. Since it cannot be assumed that this suppression mechanism must be also functional in mammal cells, the internal stop codon was deleted by site-directed mutagenesis (converting gtccgcTAGgggtgc into gtccgcgggtgc). This deletion creates a new target site for the restriction enzyme Sac II (ccgcgg) to permit a simple confirmation of success by digestion; the resulting mutant was named pIJO-39. The mutagenesis primers employed were i120 (gtcCGCGGGtgcttgcgg) and i121 (gcaagcaCCCGCGgacaa). In two separate PCR reactions using 250 ng pIJO-18 as the template in 50 μl reactions, amplification products were obtained from i116 (ATGGGCGGTAGGCGTGTA) and i121 as well as i117 (CAGGTTCAGGGGGAGGTG) and i120 (both programs: 20 cycles each with 92° C. for 15 seconds, 56° C. for 30 seconds and 72° C. for 2 min; Pwo DNA polymerase (Roche Molecular Biochemicals, Sandhofer Straβe 116, D-68305 Mannheim, Germany), 2 mM MgSO₄; the primers i116 and i117 bind outside the RdRp in the plasmid). These two amplification products were purified by agarose gel electrophoresis and commonly employed as templates in a PCR reaction with primers i116 and i117 with the same thermocycler program as in the two half reactions. Successful introduction of the mutaenized sequence (contained in the complementary primers i120 and i121) was checked and confirmed by restriction with Sac II and sequencing (annexed as Sequence #1, see also FIG. 2).

2. Cloning of Substrate RNA

SatC was converted into cDNA with primer i109 (GGGCAGGCCC) from the above described total RNA using SuperScript II reverse transcriptase and amplified with primers i110 (cccgggcaggccccc) and i112 (cccgggataactaagggtttcatac) from the cDNA mixture as described for the RdRp. Using the uncoded adenosyl overhangs in amplification products of Taq polymerase, this product was subcloned into the vector pGEM-T (Invitrogen). Three independently obtained clones each (plasmids pIJO-04, pIJO-20 and; pIJO-21) of SatC were sequenced, which yielded significant deviations from the published sequence. However, since these changes were common to the three respective clones, they were included in the final sequence of this work (annexed as SEQUENCES #2 and #3, providing termini with restriction sites for SmaI {cccggg}; SEQUENCE #4 for pIJO-20).

In SatC, the most significant deviations of the three independent clones from the sequence #X12750 were: (1) In the viral promoter for the genomic strand, two nucleotide exchanges and one insertion were found. Thus, the sequence changes from ccGTccga to ccCCcGcga. (2) In the central region, AT-rich insertions of a total of 76 bases were unexpectedly found.

Based on the sequences obtained together with previously published work [Carpenter & Simon, 1998, Nucl. Acids Res. 26, 2426-32; and Simon et al. 1988, EMBO J 7, 2645-51] the following viral promoters were established for this work (sequences of the sense strand):

. . . tatgaaacccttagttatccc-OH, and
. . . agcctccctcctcgcgggggggggcctgccc-OH

The following plasmids were generated for expression of the substrate transcript of the RdRp:

pIJO-60 and pIJO-61: A 1334 bp EcoRI/EcoRI fragment containing EMCV IRES followed by GFP from pIJO-17 was transferred into the BstE II site within the SatC region of pIJO-20; the termini of the insert and vector were filled by treatment with Klenow polymerase prior to ligation. This ligation generated pIJO-27 (IRES/GFP co-linear with SatC) and pIJO-28 (IRES/GFP anti-parallel to SatC). These two SatC cassettes with the IRES/GFP insert were isolated with Sma I as 1735 bp fragments and inserted into the Sma I site of pIJO-24. pIJO-24 is a vector in which the Sma I site is followed on the 5′ side by a CMV (Cytomegalovirus) and T7 promoter and on the 3′ side by the ribozyme of the hepatitis delta virus, the T7 terminator and the HSV thymidine kinase polyadenylation signal. After the processing by the ribozyme, the TCV-RdRp should dispose of exact (wild-type) termini in the primary transcript. This cloning generates a total of four possible combinations, two of which are relevant for these experiments: pIJO-60 expresses SatC in sense and IRES/GFP in antisense orientation; pIJO-61 expresses both SatC and IRES/GFP in antisense orientation. The antisense orientation of the IRES/GFP cassette ensures that the cell can express the GFP reporter only after successful transcription from the primary transcript by the RdRp.

The IRES upstream from GFP is mainly intended for enabling the expression of GFP in the construct pIJO-60 by circumventing three natural ATG codons upstream from the gene for the reporter (see SEQUENCE #2). However, since the influence of the secondary structure of an IRES on the processivity by the RdRp cannot be predicted, further constructs have been established for the provision of substrate RNA without IRES. In the two plasmids pIJO-79 and pIJO-80, GFP is also contained in antisense orientation in the primary transcript. As the starting point, only the expression of SatC was chosen in antisense orientation this time, hoping that, as with other RNA viruses, an asymmetric replication of the two strands occurs in TCV, i.e., a relative enrichment of the strand being in genomic orientation. The cause of this observation resides in the fact that the promoter on the antisense strands is stronger, and thus a better detection of reporter expression from the primary transcript having this orientation should be possible. Another advantage of the antisense strand is the short leader free from AUG codons upstream from the BstE-II site: in the plus strand, start codons would be present upstream from GFP, so that an IRES should not be omitted in the cloning as herein described.

For the preparation of pIJO-79 (SEQUENCE #5) and pIJO-80, GFP as a Klenow-treated Nco I/Xba I fragment from pEGFP (Clontech) was inserted into pIJO-20 pretreated with BsteE II and Klenow, to form pIJO-78. The GFP/SatC cassette was inserted as a 1139 bp Sma I fragment between the (as compared to CMV, weaker) hPGK promoter and the CMV polyA signal to form pIJO-80. For the plasmid pIJO-79, the Sma I fragment was inserted between the T7 promoter and the HDV ribozyme/T7 terminator.

Expression of the primary transcript by co-transfection with T7 RNA polymerase allows expression especially when a potential polyA signal (AATAAA) should be active for the polymerase II in the antisense strand of SatC (see SEQUENCE #3 in the annex).

3. Modifications of the RdRp

In the sequence examinations, we were surprised to find an internal. ATG (at position 811; highlighted by capital letters in SEQUENCE #1) in very good Kozak context [Kozak 1989 in J Cell Biol 108, 229-41] 60 bp downstream from the mutagenized TAG stop codon.

| | |
|---|---|
| Kozak: | gccgccgccAUGg (or gccgccaccAUGg) |
| TCV RdRp: | aacccggccAUGc |

Although the literature [White et al., 1995 in Virology 211, 525-34] describes the expression of the RdRp as a 88 kD protein in a fusion with P28, we consider it consistent with the as yet known data that the RdRp may also be expressed as an about 58 kD protein after re-initiation of translation after the stop at P28, and possibly it is just this gene product which is the active RdRp. Therefore, we have prepared an additional expression plasmid for TCV-RdRp, pIJO-83, which only expresses the carboxyl-terminal fragment of 1515 bp (505 amino acids) with a theoretical molecular weight of 58 kD. For the preparation of pIJO-83, a Sac II/Not I fragment from pIJO-39 (the TAG-deleted mutant) was transferred into the same restriction sites of the vector pEGFP-N1 as a substitute for GFP. The Sac II site was generated by the deletion of the internal stop codon in the cloning of pIJO-39 from pIJO-18.

4. Transfection of Expression Plasmids

Plasmid pIJO-39 (TCV 88 kD protein) was co-transfected with SatC expression plasmids pIJO-60, pIJO-61, pIJO-79 or pIJO-80 in 5×10⁵ 293 cells with Polyfect (Qiagen); in transfections of SatC plasmids with a T7 promoter, an expression plasmid for T7 RNA polymerase under the control of the CMV promoter was additionally included in the transfection mixture (i.e., for pIJO-60, pIJO-61 and pIJO-79).

In other experiments, plasmid pIJO-83 (TCV 58 kD protein) was co-transfected with SatC expression plasmids in 293 cells as described for pIJO-39.

In yet other experiments, combinations of pIJO-83 and pIJO-18 (the wild-type RdRp sequence with a stop codon downstream from the 28 kD gene) were co-transfected in 293 cells together with SatC expression plasmids.

Negative controls consisted of transfections where the expression plasmid for the RdRp was replaced with a plasmid that expresses no proteins in mammal cells (pUC-19 or pBluescript).

The expression of the reporter protein was observed by fluorescence microscopy with an excitation of 470 to 490 nm and cut-off of 515 nm.

### LEGEND TO THE FIGURES

FIG. 1 shows a schematic comparison of amplification of the primary transcript, coupled amplification/replication, and amplification coupled to asymmetrical replication. The primary transcript (a) with foreign gene in antisense (gray arrow) and viral promoter (VP) is recognized and transcribed into secondary copies by the RdRp (circle); promoters which are in the wrong orientation and therefore cannot be recognized are symbolized by the rotated sequence of letters “VP” (b). If the primary transcript bears a viral promoter at the 5′ end, the secondary transcript can serve as a substrate for other antisense transcripts (c), which again generate sense RNA. For asymmetric replication, the primary transcript has two internal VPs which frame a gene of the bicistronic primary RNA. The second gene (represented with dashes) is expressed from a transcript which initiates in the internal promoter in the primary transcript. In this example, it cannot be replicated since a VP has been omitted at the 5′ terminus of the primary transcript. However, the small transcript which issues from the secondary transcript by internal initiation bears VPs at both termini and can thus be replicated/amplified (d).

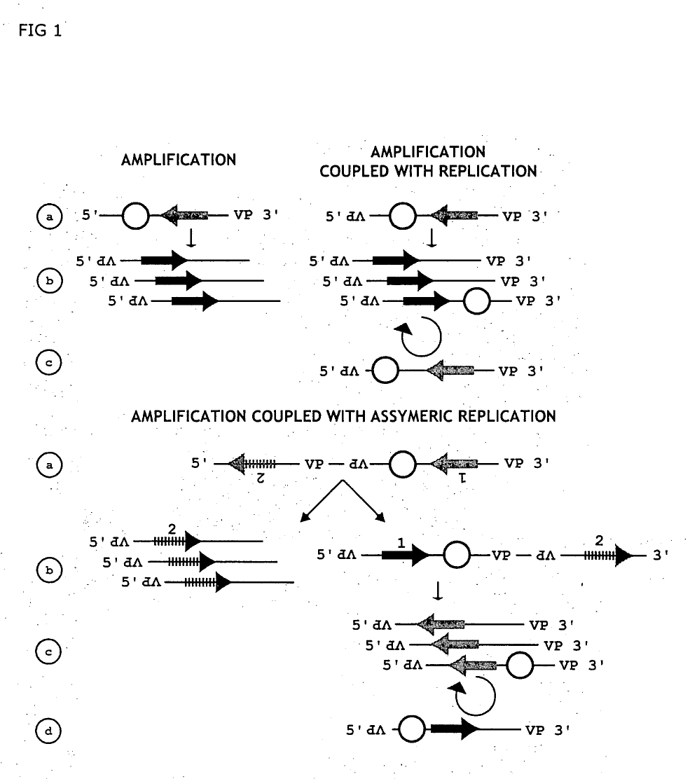

In this Example, the RdRp itself is not coded by the primary transcript in such a way that it is enclosed by viral promoters on both sides. Thus, in contrast to conventional viral replication, the system is shut down when the RdRp expression is shut off. In addition, in the Examples of FIG. 1, the foreign gene can be expressed only from secondary transcripts because it is present in a non-coding orientation on the primary transcript.

FIG. 2 shows a schematic presentation of the cloned genes for the TCV RdRp as compared with the published sequence #M22445 in GenBank. Differences between #M22445 and the corrected sequence of this work are shown by vertical ticks in the boxes; differences marked with an asterisk (*) yield changes in the amino acid sequence. To conclude: pIJO-38 bears the wild-type TCV RdRp gene, pIJO-39 bears a specific deletion of an internal stop codon, and pIJO-83 bears an amino-terminal deletion of the 88 kD protein. The translation of the 58 kD protein from pIJO-83 starts with an internal ATG downstream from the stop codon for the 28 kD protein.

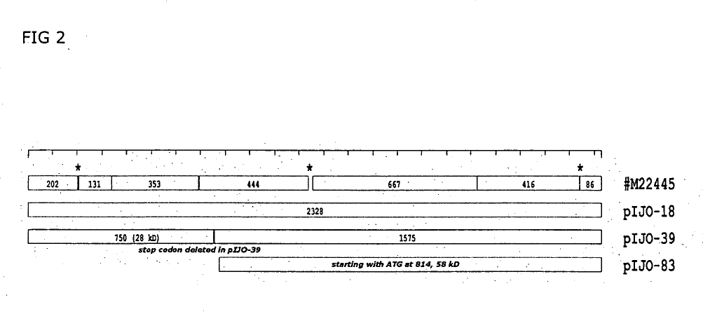

List of Abbreviations

Age I restriction enzyme

ATG codon for initiation of translation

AUG codon for initiation of translation, RNA sequence

BstE II restriction enzyme

Cap- methyl-GpppG-5′-terminal modification of mRNA

CMV cytomegalovirus

cDNA complementary DNA, copy DNA

DNA deoxyribonucleic acid

DSZM Deutsche Sammlung von Mikroorganismen und Zellkulturen of Braunschweig

EGFP enhanced green fluorescent protein, reporter protein

GFP green fluorescent protein

hCMV IE human cytomegalovirus, immediate early promoter

HDV hepatitis delta virus

hPGK gene of the human phosphoglycerate kinase

HSV herpes simplex virus

IkB inhibitor kappa B

IRES internal ribosomal entry site

kD kilo-Dalton, 1000 g/mol

mRNA messenger RNA

P88 88 kD gene product, potential RdRp of TCV

PCR polymerase chain reaction

PGK phosphoglycerate kinase

Pwo Pyrococcus woesei

pUC plasmid pUC

RdRp(s) RNA-dependent RNA polymerase

RNA ribonucleic acid

Sac II restriction enzyme

Sat satellite

SatC satellite. RNA C

Sma restriction enzyme

ssRNA single stranded RNA

TAG DNA sequence for stop codon

TCV turnip crinkle virus

tRNA transfer RNA

VP(s) viral promoter(s)

1-31. (cancelled)

32. A method for the amplification of nucleic acids in animal cells, which comprises introducing
(i) an RNA-dependent RNA polymerase (RdRp) of a plant virus and
(ii) an RNA which contains one or more promoters or cis-active signals into animal cells:

33. The method of claim 32, wherein said animal cells are selected from mammalian cells, insect, worm and amphibian cells.

34. The method of claim 33, wherein said mammalian cells are human cells, with the exception of embryonal stem cells.

35. The method of claim 32, wherein said RdRp is normally active in plant cells and whose gene can be obtained from plant cells.

36. The method of claim 35 wherein said RdRp has the sequence #1.

37. The method of claim 32, wherein said RdRp is introduced into said animal cells as an expressible gene.

38. The method of claim 32, wherein the activity of said RdRp is modulated by changes in the culturing temperature.

39. The method of claim 32, wherein the toxicity of the RdRp is reduced by mutagenesis in combination with selection.

40. The method according to claim 32, wherein said one or more promoters recognized by the RdRp are introduced into the cell in addition to the RdRp gene.

41. The method according to claim 32, wherein said one or more promoters recognized by the RdRp are synthesized as primary transcripts in the cell.

42. The method according to claim 40, wherein said RdRp is encoded by a separate transcript separately from its promoter.

43. The method of claim 41, wherein the gene of said RdRp is part of the primary transcript.

44. The method of claim 41, wherein the primary transcript is synthesized within the animal cell by a cellular polymerase.

45. The method of claim 44, wherein the cellular polymerase is a RNA polymerase II, or a recombinantly introduced polymerase.

46. The method of claim 40, wherein the strength of naturally occurring promoters for the RdRp is modified by mutagenesis.

47. The method according to claim 32, wherein the occurrence of the amplification of an RNA which codes for a foreign gene possesses a function selected from the group consisting of acting as an antisense transcript of a cellular transcript, serving as a genomic RNA for the preparation of recombinant viruses, and having itself enzymatic activity.

48. The method according to claim 47, wherein said foreign gene is in antisense orientation followed by at least one promoter in coding orientation in the primary transcript, so that the foreign gene cannot be expressed without the RdRp, but is activated by its expression.

49. The method of claim 47, wherein the RNA amplified by the RdRp contains functional sequences selected from the group consisting of amplified RNA IRES elements, shunt donor and acceptor, and translation enhancers for improving foreign gene expression.

50. The method of claim 47, wherein the RNA amplified from the RdRp contains at least one polyadenylation tract.

51. The method of claim 47, wherein the RNA amplified from the RdRp is processed by a ribozyme.

52. The method of claim 47, wherein the RNA amplified from the RdRp contains signals for packaging into a viral envelope and is packaged into viral envelopes.

53. The method according to claim 32, characterized in that said RNA contains two similar promoters in opposite orientation which initiate the synthesis from both RNA strands to induce an enhanced amplification.

54. The method according to claim 32, characterized in that said RNA contains two dissimilar promoters in opposite orientation which initiate the synthesis from both RNA strands to induce an enhanced amplification.

55. The method according to claim 41, wherein the expression of the primary transcript is regulated by a system based on cellular RNA polymerase II.

56. The method according to claim 41, wherein the expression of the RdRp is regulated by a system based on cellular RNA polymerase II.

57. The method according to claim 41, wherein the expression of the primary transcript and of the RdRp is regulated by a system based on cellular RNA polymerase II.

58. The method according to claim 32 which is employed for finding favorable mutations in the RdRp or in the foreign gene.

59. The method of claim 58, wherein said favorable mutations cause an effect selected from the group consisting of an improved replication of the system, an improved performance of the foreign gene and a lower toxicity of the foreign gene.

60. The method of claim 32 which is switched on or activated by cellular events.

61. The method of claim 60, wherein the activation has an effect selected from the group consisting of causing expression of a foreign gene being effected through a fusion protein at the RdRp, serving for the detection of signal transduction pathways or the translocation of intracellular factors, being effected by the entering of a diffusible substance or a toxin, and being effected through infection of the host cell.

62. The method according to claim 40, wherein the RdRp and the promoters are derived from a plant virus.

63. The method of claim 62, wherein a polymerase from a member of the Tombusviridae family is used, and a modified satellite RNA is used as the amplified RNA.

64. The method of claim 62, wherein the polymerase of turnip crinkle virus is used, and a modified satellite RNA of turnip crinkle virus or a modified genomic RNA of turnip crinkle virus is used as the amplified RNA.

65. The method of claim 32 which is applicable in vitro in animal cells.

66. The method of claim 32 which is applicable in vivo in organisms selected form the group consisting of mammals (except for humans), insects and worms.

67. The method according to claim 32, wherein the gene of the RdRp is part of its own transcript.

68. The method of claim 66, wherein the foreign gene of the RdRp or at least one promoter in the primary transcript are in antisense orientation and said foreign gene cannot be expressed by the cell without said RdRp, but is translated in the presence of said RdRp.

69. The method according to claim 32, wherein the amplified RNA of the RdRp is processed by a ribozyme;

70. The method according to claim 32, wherein the amplified RNA of the RdRp contains signals for packaging into a viral envelope and is packaged into viral envelopes.

71. The method according to claim 32, wherein the amplified RNA of the RdRp codes for a gene or several genes.

72. The method according to claim 32, wherein the amplified RNA of the RdRp codes for genes in different orientations.

73. The method according to claim 66, wherein the amplified RNA of the RdRp represents an RNA which displays its action in cells without translation.

74. The method according to claim 32, wherein the amplified RNA of the RdRp codes for a genomic or subgenomic RNA of another virus.

75. The method according to claim 32, wherein the primary transcript is synthesized by a bacterial RNA polymerase.

76. The method according to claim 75, wherein the primary transcript is synthesized by a bacterial RNA polymerase selected from the group consisting of the bacterial RNA polymerases T7, SP6 and T3 within the mammal cell.

77. An animal cell, which is obtainable by a method according to claim 32.

78. A transcript, which is suitable for introducing an RNA-dependent RNA polymerase (RdRp) of a plant virus and an RNA which contains one or more promoters or cis-active signals into animal cells according to the method of claim 32.

79. The transcript of claim 78, which contains an RdRp which is encoded by a transcript separately from its substrate RNA.

80. The transcript of claim 78, wherein the gene of the RdRp is part of the primary transcript.

81. The transcript according to claim 78, which contains naturally occurring promoters which were subjected to mutagenesis.

82. The transcript according to claim 78, which contains a foreign gene in antisense orientation followed by at least one promoter in coding orientation in the primary transcript, so that the foreign gene cannot be expressed without said RdRp, but is activated by its expression.

83. The transcript according to claim 78, the activity of which being modulated by changing the culturing temperature.

84. The transcript according to claim 78, the toxicity of which being reduced by mutagenesis in combination with selection.

85. The transcript according to claim 78, wherein the RdRp and promoter(s) are derived from a plant virus.

86. The transcript according to claim 78, wherein the polymerase is derived from a member of the Tombusviridae family.

87. The transcript of claim 86, wherein the polymerase is derived from turnip crinkle virus.

88. The method of claim 32 which is suitable for the amplification of nucleic acids in animal cells.

89. The method of claim 88 which is suitable for the amplification of RNA in animal cells.

90. The method according to claim 88 which is suitable for controlling gene expression.

91. The method according to claim 88, which is suitable for in vivo applications for gene therapy, vaccination and therapeutic vaccination.

92. The method according to claim 88 which is suitable for the preparation of a medicament for gene therapy, vaccination or therapeutic vaccination.

93. A test kit for determining the amplification of nucleic acids, consisting of two components (K), a cell line which bears the gene for the RdRp stably integrated in its genome (K1), and a collection of expression plasmids for the primary transcript (K2).

94. The test kit according to claim 93, characterized in that it expresses the gene constitutively or, under the control of a controllable promoter, only upon induction (K1), and preferred plasmids (K2) combine the cis-active signals for the RdRp with a region having several restriction sites for various restriction enzymes, wherein the expression plasmids dispose of promoters of different strength and polyadenylation signals for the expression of the primary transcript in the target cell, and a bacterial promoter at the beginning and a suitable restriction site for a restriction enzyme at the end of the cassette for the primary transcript.

95. The test kit according to claim 93, consisting of a cell line which bears both RdRp and the expression cassette for the primary transcript stably integrated in its genome, but with the RdRp being under the control of an inducible promoter which responds to a test substance of the user, and the primary transcript bears both a gene for the RdRp and one for a reporter gene in a replicable and amplifiable combination.
